(19) United States
(12) Patent Application Publication
(10) Pub. No.: US 2024/0366378 A1
(43) Pub. Date: Nov. 7, 2024

(54) ADJUSTABLE ANNULOPLASTY DEVICE

(71) Applicant: EDWARDS LIFESCIENCES CORPORATION, Irvine, CA (US)

(72) Inventors: Felino V. Cortez, Jr., Bowie, MD (US); Stephen Cournane, Severn, MD (US); Nancy Ling Chung, Edgewater, MD (US); Stephen Epstein, Millersville, MD (US); Luke Anthony Zanetti, Parkton, MD (US); Ashley Nicolette Hinga (formerly Keffer), Eldersburg, MD (US); Sai Prasad Uppalapati, Plano, TX (US)

(21) Appl. No.: 18/627,145

(22) Filed: Apr. 4, 2024

Related U.S. Application Data
(60) Provisional application No. 63/499,948, filed on May 3, 2023.

(51) Int. Cl. A61F 2/24

(52) U.S. Cl. CPC A61F 2/2448; A61F 2220/0016; A61F 2220/0025; A61F 2230/0034; A61F 2250/0003; A61F 2250/001

(57) ABSTRACT

A device for treating a heart valve comprising a curved frame and a covering at least partially enclosing the curved frame.

### RELATED APPLICATION

This application claims the benefit of U.S. Patent Application No. 63/499,948, filed on May 3, 2023, the entire disclosures of which are incorporated by reference in their entireties.

### BACKGROUND

### Field

The present disclosure relates to the field of medical devices and procedures.

### Description of the Related Art

An annuloplasty is a procedure to tighten or reinforce the ring (annulus) around a valve in the heart. For example, due to various factors, two or more leaflets of a heart valve may not coapt properly, resulting in regurgitation of the blood flow (e.g., backwards blood flow). An annuloplasty ring may be attached (e.g., sewn) to the annulus of the heart valve to pull the leaflets together for proper coaptation and to re-establish proper valve function.

### SUMMARY

Some implementations of the present disclosure relate to a device for treating a heart valve including: a curved frame; and a covering at least partially enclosing the curved frame.

In some aspects, the techniques described herein relate to a device, further including a first expandable bladder connected to the curved frame.

In some aspects, the techniques described herein relate to a device, further including a flow tube configured to allow inflow of a fluid into the first expandable bladder.

In some aspects, the techniques described herein relate to a device, wherein the flow tube includes a one-way valve.

In some aspects, the techniques described herein relate to a device, wherein the curved frame is D-shaped and includes a flat portion, and wherein the first expandable bladder is disposed across from the flat portion.

In some aspects, the techniques described herein relate to a device, further including a second expandable bladder.

In some aspects, the techniques described herein relate to a device, further including a first fill tube connected to the first expandable bladder and a second fill tube connected to the second expandable bladder.

In some aspects, the techniques described herein relate to a device, further including one or more eyelets connected to an inner surface of the frame.

In some aspects, the techniques described herein relate to a device, wherein the curved frame includes two or more segments interconnected by one or more joints.

In some aspects, the techniques described herein relate to a device, wherein the one or more joints are configured to facilitate bending of the curved frame.

In some aspects, the techniques described herein relate to a device, wherein the curved frame has a closed form.

In some aspects, the techniques described herein relate to a device, wherein the curved frame has an open form.

In some aspects, the techniques described herein relate to a device, wherein the curved frame includes one or more openings.

In some aspects, the techniques described herein relate to a device, further including one or more tissue anchors within the one or more openings.

In some aspects, the techniques described herein relate to a device, wherein the one or more tissue anchors include cavities configured to receive an anchor driver.

In some aspects, the techniques described herein relate to a device, further including a suture extending at least partially through the covering.

In some aspects, the techniques described herein relate to a device, wherein the suture includes two free ends configured to extend outside the covering.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements.

FIG. 1 illustrates an example representation of a heart having various features relevant to certain examples of the present inventive disclosure.

FIG. 2A provides a cross-sectional view of the left ventricle and left atrium of an example heart.

FIG. 2B illustrates a top/surgeon's view looking at the mitral valve of the heart.

FIG. 3A provides an illustration of the heart in a state where functional mitral valve regurgitation (FMR) is present.

FIG. 3B show a mitral valve where the annulus is dilated and deformed causing mitral regurgitation through the commissure.

FIG. 4 illustrates an example heart with an expandable repair device 400 implanted at a mitral valve of the heart in accordance with one or more examples.

FIG. 5 illustrates an example inflatable covering for one or more valve repair devices in accordance with one or more examples.

FIG. 6 illustrates an example support device and/or frame of an example valve repair device in accordance with one or more examples.

FIG. 7 illustrates an example valve repair device in accordance with one or more examples.

FIG. 8 illustrates an example valve repair device comprising one or more sutures and/or similar devices anchored to one or more eyelets of the device in accordance with one or more examples.

FIG. 9 illustrates another example support device of an example valve repair device in accordance with one or more examples.

FIG. 10A provides a partially cross-sectional top view of the device with cross sections through a covering to allow viewing of one or more flexible joints of the device.

FIG. 10B provides a close-up view of a joint.

FIG. 11 illustrates at least a portion of an example valve repair device comprising a ring and/or ring-shaped expandable device in accordance with one or more examples.

FIG. 12 illustrates at least a portion of an example valve repair device comprising a ring and/or ring-shaped expandable device in accordance with one or more examples.

FIG. 13 illustrates at least a portion of an example valve repair device comprising a ring and/or ring-shaped expandable device in accordance with one or more examples.

FIG. 14 illustrates at least a portion of an example valve repair device comprising a ring and/or ring-shaped expandable device in accordance with one or more examples.

FIG. 15 illustrates at least a portion of an example valve repair device comprising a ring and/or ring-shaped expandable device in accordance with one or more examples.

FIG. 16 illustrates another example valve repair device in accordance with one or more examples.

FIG. 17 illustrates an example tissue anchor configured to anchor one or more valve repair devices described herein.

FIG. 18 illustrates an example process of driving a tissue anchor into a repair device via an anchor driver in accordance with one or more examples.

FIG. 19 illustrates one or more cords (e.g., implant contraction wires) extending into an example tissue anchor and/or clip in accordance with one or more examples.

FIG. 20 illustrates driving of one or more tissue anchors using an anchor driver 2005 in accordance with one or more examples.

FIG. 21 illustrates anchoring of one or more cords using a pledget and/or one or more clips.

FIG. 22 (FIGS. 22-1, 22-2, and 22-3) is a flowchart illustrating steps of a process for delivering and/or deforming a valve repair device in accordance with one or more examples of the present disclosure.

FIG. 23 (FIGS. 23-1, 23-2, and 23-3) provides certain images depicting various features associated with steps of the process depicted in FIG. 22.

FIG. 24 illustrates an example method of suturing at or around an annulus of a valve in accordance with one or more examples.

FIG. 25 illustrates an example method of suturing at or around an annulus of a valve 6 in accordance with one or more examples.

FIG. 26 illustrates a suturing technique at one or more leaflets of a valve in accordance with one or more examples.

FIGS. 27A and 27B illustrate extension and/or titration of one or more cords extending from a leaflet through a papillary muscle and/or through a ventricle wall 18 in accordance with one or more examples.

FIG. 28 provides an outer view of an example ventricle of a heart with various sutures anchored at the walls of the ventricle in accordance with one or more examples.

### DETAILED DESCRIPTION

The headings provided herein are for convenience only and do not necessarily affect the scope or meaning of the claimed subject matter. The present disclosure relates to systems, devices, and methods to determine access for an anatomical feature based on an analysis of one or more images representing a mineral deposit.

Although certain examples are disclosed below, the subject matter extends beyond the specifically disclosed examples to other alternative examples and/or uses, and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

### Overview

As noted above, an annuloplasty procedure can be performed to tighten or reinforce the ring (annulus) around a valve in the heart. Such procedures involve attaching a structure (e.g., annuloplasty ring) to the annulus of the heart valve. Various types of annuloplasty rings have been developed to satisfy the myriad of contexts in which an annuloplasty ring may be implanted (e.g., different sized heart valves, heart valve abnormalities, physician preferences, etc.). In particular, annuloplasty rings come in different sizes, shapes, materials, suture features for attachment, and so on, which provide physicians with options to implement. In many cases, a physician can use one or more ring sizers to determine a size of an annuloplasty ring to use. The physician can overlay D-shaped plates (e.g., the ring sizers) of different sizes onto the heart valve to identify an optimal size of an annuloplasty ring for the specific heart valve.

In some cases, annuloplasty rings may be replaced and/or supplemented in subsequent procedures by additional implants. For example an annuloplasty ring may be replaced and/or supplemented with a replacement valve implant (e.g., during a transcatheter aortic valve replacement (TAVR) procedure). Some annuloplasty rings may not be designed to be replaced and/or supplemented. For example, some annuloplasty rings may be composed of generally elastic materials that may to return to a formed shape after any applied stresses are relieved and thus may not be mechanically deformed in a significant way (e.g., with a balloon and/or balloon expandable valve). Some annuloplasty rings can provide a very rigid constraint to the annulus that can prevent replacement valves from deploying in an optimal shape (e.g., a parallel shape).

This disclosure describes techniques related to annuloplasty rings having features configured to increase flexibility and/or adjustability of the annuloplasty rings. Some example annuloplasty rings can be configured for surgical delivery. In some examples, following surgical delivery of an example annuloplasty ring and/or repair device, adjustments to the annuloplasty ring and/or repair device can be performed on a beating heart via, for example, one or more sutures extending from the device to outside the body.

In some examples, an annuloplasty ring may comprise one or more segments/body portions having and/or connected by flexible joints and/or portions. Some annuloplasty rings may have a continuous (e.g., a “D-shaped structure) and/or non-continuous structure (e.g., a “C-shaped” structure) that may allow for future implanted devices to be able to expand the annuloplasty ring somewhat. The body portions, which may include any means for treating, tightening, and/or reinforcing an annulus portion of a heart valve, and/or deformable portions may be configured to form a continuous and/or non-continuous implant which may be configured to be attached to an annulus portion of a heart valve.

### Example Heart Anatomy

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary vein, aorta, etc.).

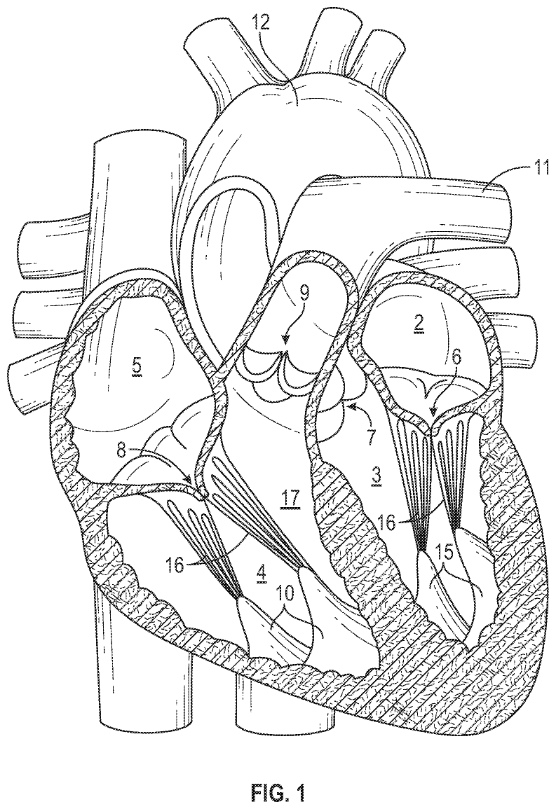

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (e.g., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior and septal leaflets of the tricuspid valve, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

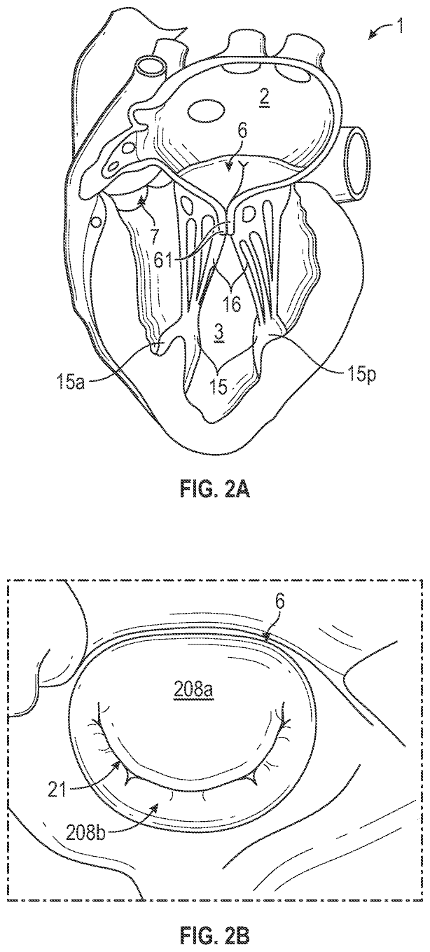

FIG. 2A provides a cross-sectional view of the left ventricle 3 and left atrium 2 of an example heart 1. While some example devices and/or methods are described herein with respect to the left ventricle 3, mitral valve 6, and/or left atrium 2, such devices and/or methods may be applied to and/or performed within other areas of the heart, including the right ventricle, right atrium, and/or tricuspid valve. The diagram of FIG. 2A shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2A and various other figures provided herewith and described herein in the context of certain examples of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve 6 as shown in FIG. 2A, the valve leaflets may extend inward from the valve annulus and come together in the flow orifice to permit flow in the outflow direction (e.g., the downward direction in FIG. 2A) and prevent backflow or regurgitation toward the inflow direction (e.g., the upward direction in FIG. 2A). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendineae 16 to each valve leaflet (e.g., the anterior and posterior leaflets). With respect to the state of the heart 1 shown in FIG. 2A, the proper coaptation of the valve leaflets, which may be due in part to proper position of the papillary muscles 15, may advantageously result in mitral valve operation substantially free of leakage.

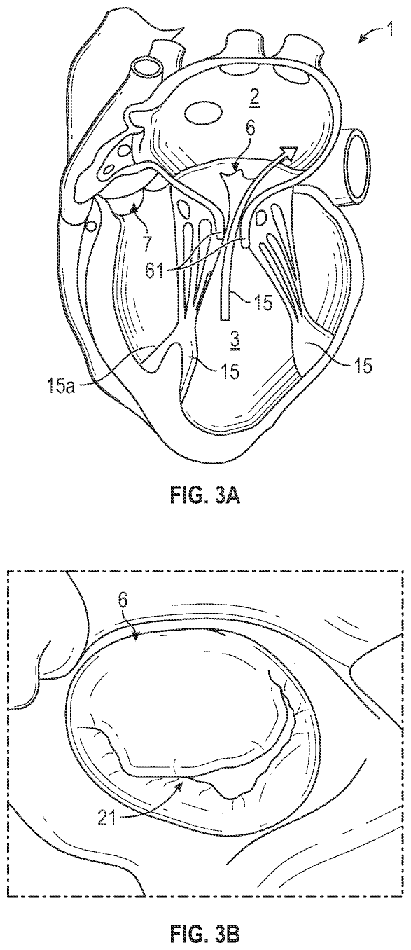

Heart valve disease represents a condition in which one or more of the valves of the heart fail to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3A, which illustrates a cross-sectional view of a heart 1 experiencing mitral regurgitation flow 20, dilation of the left ventricle may cause changes in the position of the papillary muscles 15 that allow flow 20 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

FIG. 2B illustrates a top/surgeon's view looking at the mitral valve 6 of the heart 1. The mitral valve 6 generally includes an anterior leaflet 208a that is relatively large and attaches to the anterior segment of the annulus, while a posterior leaflet 208b is smaller but extends further circumferentially and attaches to the posterior segment of the annulus, as shown in FIG. 2B. The anterior leaflets 208a and the posterior leaflets 208b join and insert into the annulus at the commissures 21, namely the anterior commissure and posterior commissure.

### Example Mitral Valve Conditions

Several diseases can affect the structure and function of the mitral valve. The mitral valve and, less frequently, the tricuspid valve, are prone to deformation and/or dilation of the valve annulus, tearing of the chordae tendineae, and/or leaflet prolapse, which results in valvular insufficiency wherein the valve does not close properly and allows for regurgitation or back flow from the left ventricle into the left atrium. Deformations in the structure or shape of the mitral or tricuspid valve can be repairable.

Mitral regurgitation is one of the most common valvular malfunctions in the adult population, and typically involves the elongation or dilation of the posterior two-thirds of the mitral valve annulus, the section corresponding to the posterior leaflet. The most common etiology of systolic mitral regurgitation is myxomatous degeneration, also termed mitral valve prolapse (29% to 70% of cases), which afflicts about 5 to 10 percent of the population in the U.S. Women are affected about twice as often as men. Myxomatous degeneration has been diagnosed as Barlow's syndrome, billowing or ballooning mitral valve, floppy mitral valve, floppy-valve syndrome, prolapsing mitral leaflet syndrome, or systolic click-murmur syndrome. The symptoms include palpitations, chest pain, syncope or dyspnea, and a mid-systolic click (with or without a late systolic murmur of mitral regurgitation). These latter symptoms are typically seen in patients with Barlow's syndrome, where extensive hooding and billowing of both leaflets are the rule. Some forms of mitral valve prolapse seem to be hereditary, though the condition has been associated with Marfan's syndrome, Grave's disease, and other disorders.

Myxomatous degeneration involves weakness in the leaflet structure, leading to thinning of the tissue and loss of coaptation. Barlow's disease is characterized by myxoid degeneration and appears early in life, often before the age of fifty. In Barlow's disease, one or both leaflets of the mitral valve protrude into the left atrium during the systolic phase of ventricular contraction. The valve leaflets are thick with considerable excess tissue, producing an undulating pattern at the free edges of the leaflets. The chordae are thickened, elongated and may be ruptured. Papillary muscles are occasionally elongated. The annulus is dilated and sometimes calcified. Some of these symptoms are present in other pathologies as well and, therefore, the present application may refer to myxoid degeneration, which is the common pathologic feature of the various diagnoses, including Barlow's syndrome.

Other causes of mitral regurgitation include ischemic heart disease with ischemic mitral regurgitation (IMR), dilated cardiomyopathy (in which the term “functional mitral regurgitation FMR is used), rheumatic valve disease, mitral annular calcification, infective endocarditis, fibroelastic deficiency (FED), congenital anomalies, endocardial fibrosis, and collagen-vascular disorders. IMR is a specific subset of FMR, but both are usually associated with morphologically normal mitral leaflets. Thus, the types of valve disease that lead to regurgitation are varied and present vastly differently.

FIG. 3A provides an illustration of the heart 1 in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIGS. 3A and 3B may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3A, the leaflets 61 of the mitral valve 6 (or tricuspid valve) are not in a state of coaptation, resulting in an opening between the mitral valve leaflets 61 during the systolic phase of the cardiac cycle, which allows the leakage flow 20 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3A) and/or unwanted backflow or regurgitation toward the inflow direction (e.g., the downward direction in FIG. 3A).

FIG. 3B show a mitral valve 6 where the annulus is dilated and deformed causing mitral regurgitation through the commissure 21. At a structural level, four general types of structural changes of the mitral valve apparatus can cause regurgitation: leaflet retraction from fibrosis and calcification, annular dilation, chordal abnormalities (including rupture, elongation, shortening, or apical tethering or “tenting” as seen in FMR and IMR), and possibly papillary muscle dysfunction.

### Valve Repair Devices and Procedures

Various techniques/procedures may be used to repair diseased or damaged heart valves, such as mitral and tricuspid valves. These include, but are not limited to, annuloplasty (e.g., contracting the valve annulus to restore the proper size and/or shape of the valve), quadrangular resection of the leaflets (e.g., removing tissue from enlarged or misshapen leaflets), commissurotomy (e.g., cutting the valve commissures to separate the valve leaflets), shortening and transposition of the chordae tendineae, reattachment of severed chordae tendineae or papillary muscle tissue, and decalcification of valve and annulus tissue.

In some mitral valve repair procedures, mitral annuloplasty procedure (MAP) can be combined with mitral valve leaflet repair and chordal replacements. The purpose of the MAP can be to prevent re-dilation of the annulus and/or secure sufficient leaflet coaptation that affects valve durability. The procedure of sizing repair devices (e.g., annuloplasty rings) can be important. If a device is too large size, the valve may be incompetent. If the device is too small size, systolic anterior motion (SAM) and/or functional mitral stenosis (FMS) may occur. Measuring an area or the distance of an annuloplasty ring by placing a pre-shaped platform to a floppy native valve can be uncertain and/or can depend on the surgeon's experience and intuition.

Some approaches to sizing an annuloplasty ring may not guarantee accuracy. Trial-and-error sizing techniques can be imprecise, tedious, and time-consuming. An annular support ring implant as part of Degenerative Mitral Regurgitation (DMR) repair cases may be configured to stabilize the repair, but it can be difficult for surgeons to accurately predict the ring size implanted on a flaccid (e.g., arrested) heart that may be optimal once the patient is weaned off of by-pass. Sizing annuloplasty rings and/or assessment of chordal length done in a static heart can be a challenge. Inasmuch as there may be post-implant control in place (e.g., based on ventricular saline injection and/or ink test for direct view of valve competence), it may not always be reliable as the control assesses coaptation during non-physiologic conditions.

Some example methods and/or devices described herein may be configured to offer real-time annuloplasty ring and/or chordal adjustment techniques that are technically feasible. The modes of repair described herein can advantageously provide solutions for controlling optimal sizing of annular support and/or chordal repair in on-pump mitral cases to reduce the rate of post-operative complications and/or improve outcomes.

The devices and/or methods described herein can provide adjustability to repair devices and/or systems, which can have various advantages. For example, such devices and/or methods can offer real-time chordal adjustment in a beating heart and/or under physiologic loading conditions to enhance chances of a successful repair. Moreover, some examples may be configured to minimize risk and/or reduce postoperative complications.

In some examples, an annuloplasty repair (e.g., ring) device may be adjustable in situ to facilitate controlling a size of the annulus in a beating heart and/or under echo guidance that can minimize the risk of under-sizing. Some devices can be configured to expand for stability and/or support. In some examples, a repair device can comprise one or more elastic (e.g., semi-rigid) portions while allowing for some degree of annular expansion.

The ability for chordal adjustment can prevent having to estimate the appropriate neochordal length during implantation. Neochords may be inserted to the head of a papillary muscle and/or may be anchored to the external surface of one or more heart adjustments. Some examples may comprise accessory chord papillary muscle implants configured to move and/or relocate one or more papillary muscles to further improve the leaflet coaptation surface.

Some example devices and/or methods may be configured to provide optimum adjustment under echocardiographic guidance based on the reduction of regurgitation. Some examples may be configured to enhance surgical procedure by providing surgeons more control and/or fine-tuning capabilities to enhance repair.

Although various techniques/procedures are discussed herein in the context of mitral valves, the techniques/procedures can be applicable to other types of heart valves and/or anatomical structures/features.

In some examples, annuloplasty rings and/or other implants described herein may be configured for delivery and/or attachment to an annulus portion of a heart valve to treat the heart valve. An implant may be sized slightly smaller than a distended annulus.

Various implants described herein may comprise inner body portions at least partially encapsulated and/or covered by an outer sheath and/or multiple outer sheath portions. In some examples, one or more body portions of an implant may comprise a relatively rigid and/or elastic inner structural support at least partially surrounded by a pliable core material and/or a cover composed of fabric and/or other materials. For example, an implant (e.g., an annuloplasty ring) may comprise an inner skeleton of one or more bands of relatively rigid and/or elastic material (e.g., titanium, cobalt-chromium (e.g., ELIGLOY® alloy), nitinol) surrounded by a suture-permeable core material (e.g., silicone) and/or an outer fabric cover. In some examples, the inner skeleton may comprise multiple bands separated by plastic and/or other relatively low friction material (e.g., PTFE (e.g., TEFLON® PTFE)) to allow the bands to more easily flex with respect to one another.

In some examples, an implant (e.g., an annuloplasty ring) may be configured to implanted at an annulus using any of a variety of attachment means, which can include one or more sutures. For example, one or more sutures may be distributed around body portions of the implant and/or may be tied off to present minimal surface roughness and/or to reduce the chance of thrombi forming thereon.

The inner structural support and/or pliable core material of one or more body portions of an implant may be relatively rigid and/or may be configured to initially resist deformation when subjected to the stress imparted thereon by the valve annulus of an operating human heart and/or by various medical devices (e.g., balloon expandable devices). The term “deformation” is used herein in accordance with its plain and ordinary meaning and may refer to substantial permanent deformation from a predetermined manufactured and/or shape-set shape. A number of generally rigid materials can be utilized, including various bio-compatible polymers, metals, and/or alloys. An implant may comprise one or more deformable portions which may be configured to deform in response to pressure from a medical device and/or growth of a native heart valve.

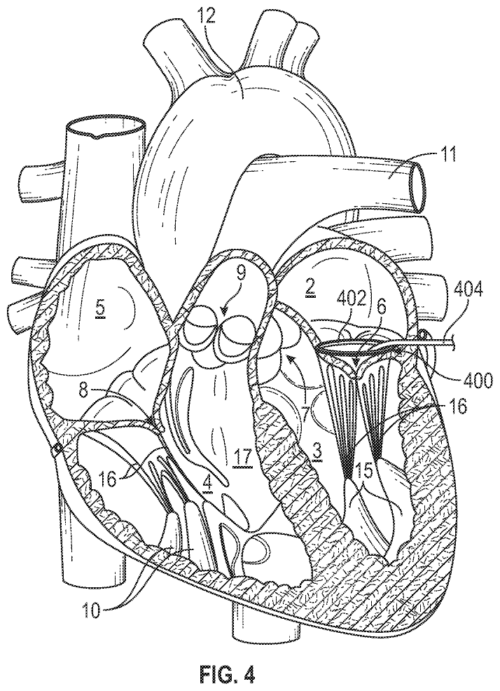

FIG. 4 illustrates an example heart 1 with an expandable repair device 400 implanted at a mitral valve 6 of the heart 1 in accordance with one or more examples. In some examples, the device 400 may comprise an expanding annuloplasty ring and/or similar device. The device 400 may be configured to achieve a desired size and/or amount of annular support in on-pump mitral repair systems to reduce rate of post-operative complications.

The device 400 may be configured to provide different flexibilities at various points about the circumference of the valve (e.g., mitral valve 6) annulus. While some annuloplasty rings may have fixed shapes and/or sized and/or may be configured for placement in a flaccid heart, the device 400 may advantageously be adjustable and/or implantable in a beating heart.

In some examples, the device 400 may comprise various features configured to prevent over-tightening and/or over-expansion of the device. If replacement valves are tightened too much, it can result in mitral stenosis and/or systolic anterior motion of the valve, which can cause outflow tract obstruction.

The device 400 can comprise one or more flexible joints interconnecting one or more segments of the device 400. The device 400 may comprise a covering (e.g., flexible material) at least partially enclosing the segments and/or fitting snugly about the segments. The joint structure may provide a mechanism for adjusting the circumference of the device 400. In some examples, the device 400 can comprise multiple flexible joints.

In some examples, the device 400 can be composed of one or more materials with varying degrees of flexibility to allow the individual segments to move independently of each other. A segment can comprise multiple different core materials (e.g., rigid frames for anterior and posterior segments and/or flexible frames for the lateral and medial segments).

The device 400 can be structured in different geometries. For example, the device 400 can have an open (e.g., C-shape) or closed (e.g., circular, D-shaped, C-shaped, etc.) shape. The device 400 may be shaped to improve efficacy in sizing and/or can provide an ability to go up in size. The device 400 may be configured to be adjustable to increase and/or decrease in circumference, diameter, and/or width. Circumferential and/or regional expansion of the device 400 can maintain the physiological shape of the annulus of the valve.

The device 400 can be delivered to the valve via any suitable means. For examples, the device 400 can be delivered surgically via a full and/or mini sternotomy.

In some examples, the device 400 may comprise an inflation tube 404 configured to couple to and/or extend from a ring 402 of the device 400.

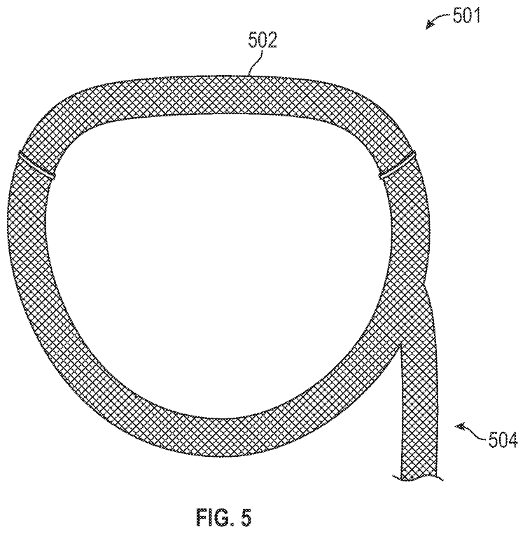

FIG. 5 illustrates an example inflatable covering 501 for one or more valve repair devices in accordance with one or more examples. The covering 501 may comprise one or more fillable chambers configured to be filled with one or more gases and/or fluids. In some examples, the covering 501 may comprise a ring 502 configured to at least partially enclose a ring-shaped portion of a frame of valve repair device. The covering 501 may further comprise a tube 504 portion configured to at least partially enclose an inflation tube of the valve repair device.

The covering 501 may be configured to be at least partially fluid-tight and/or may be inflatable. In some examples, the covering 501 may be configured to at least partially enclose a supporting ring anchored to an annulus. The covering 501 may be configured to be filled with one or more fluids (e.g., a self-curing and/or hardening polymer) via a fill tube to adjust for appropriate ring size. In some examples, the covering 501 may comprise one or more fillable sections and/or chambers. The covering 501 may be composed of one or more fabrics and/or other materials.

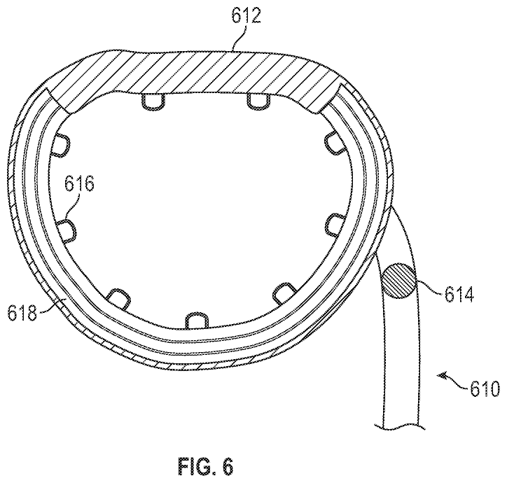

FIG. 6 illustrates an example support device and/or frame of an example valve repair device in accordance with one or more examples. In some examples, the device can comprise a generally rigid outer ring 612 providing a structure for the device. The device can further comprise an inflatable bladder 618 configured to be filled with one or more fluids (e.g., a self-curing and/or hardening polymer) via a fill tube 610 to adjust for appropriate ring size. In some examples, the fill tube 610 can comprise one or more valves 614 (e.g., one-way valves) configured to allow flow into the inflatable bladder 618 and/or configured to prevent outflow from the inflatable bladder 618. The device can comprise one or more eyelets 616 configured to mount one or more sutures and/or similar devices.

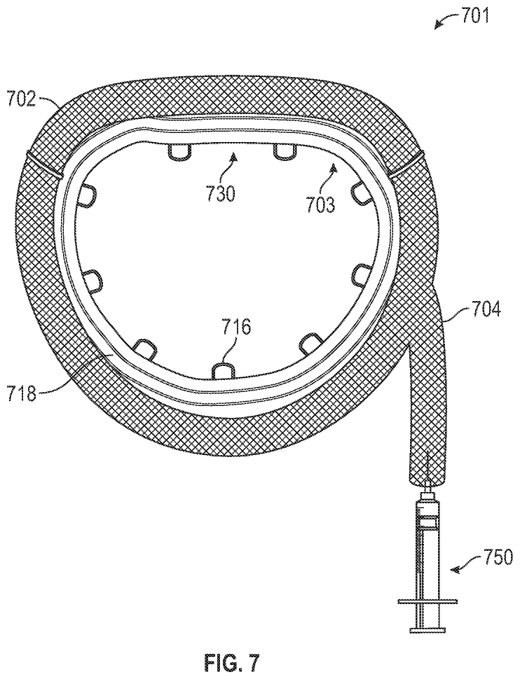

FIG. 7 illustrates an example valve repair device in accordance with one or more examples. The device can comprise a covering 701 at least partially enclosing a support device 703 of the device. The support device 703 can comprise a generally rigid outer ring providing a structure for the device 703. The device 703 can further comprise an inflatable bladder 718 configured to be filled with one or more fluids (e.g., a self-curing and/or hardening polymer) via a fill tube 704 to adjust for appropriate ring size. In some examples, the fill tube 704 can comprise one or more valves (e.g., one-way valves) configured to allow flow into the inflatable bladder 718 and/or configured to prevent outflow from the inflatable bladder 718. The device 703 can comprise one or more eyelets 716 configured to mount one or more sutures and/or similar devices.

The covering 701 may comprise one or more fillable chambers configured to be filled with one or more gases and/or fluids. In some examples, the device may comprise a ring 702. The device may further comprise an inflation tube 704. The inflation tube 704 may be configured to be filled via a syringe 750 and/or similar device.

The inflatable bladder 718 may not extend around a complete circumference of the ring 702 and/or may comprise one or more portions that are not inflatable. For example, an anterior annulus portion 730 of the device may not be inflatable. In some examples, the inflatable bladder 718 may be configured to implanted oversized and/or may be at least partially flexible and/or configured to expand inwardly to reduce a size of the annulus as desired.

The device may be configured to implanted on-pump and/or may be adjusted in a beating heart (e.g., off-pump). Filling of the device and/or inflatable bladder 718 may be configured to performed with imaging (e.g., echo imaging) to enable filling to desired ring 702 size to create a desired level of leaflet coaptation and/or restoration of the valve to a more functional state. The ring 702 and/or inflatable bladder 718 may be configured to expanded and/or to pull in the annulus of the valve.

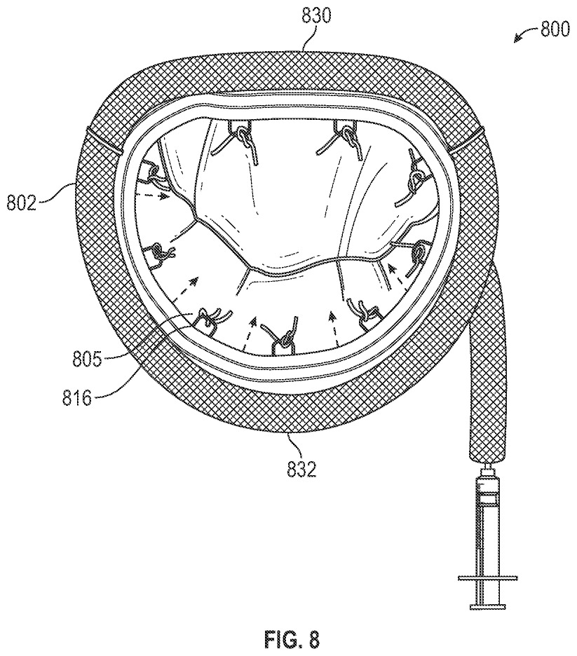

FIG. 8 illustrates an example valve repair device 800 comprising one or more sutures 805 and/or similar devices anchored to one or more eyelets 816 of the device 800 in accordance with one or more examples. In some examples, the one or more sutures 805 may be configured to attach to an inner side of a ring 802 of the device 800. Inflation of the ring 802 can be configured to move a posterior annulus portion 832 inwardly (e.g., towards an anterior annulus portion 830).

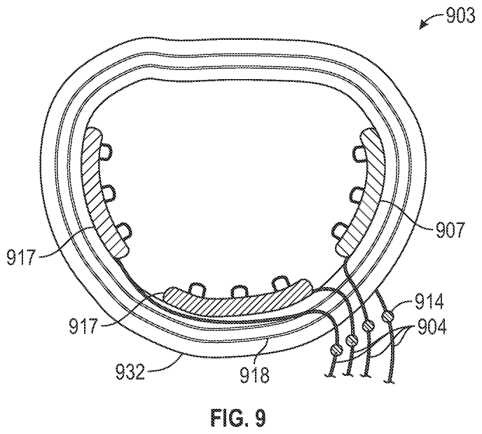

FIG. 9 illustrates another example support device 903 of an example valve repair device in accordance with one or more examples. In some examples, the device 903 may comprise multiple inflatable balloons configured to be filled with a multi-ported catheter and/or similar device. In some examples, the device 903 may comprise a primary balloon 907 and/or one or more secondary balloons 917. In some examples, the balloons may be aligned in-series along an inner surface of a posterior annulus portion 932 of the device 903. The balloons may be configured to form one or more residual gaps and/or a non-coapting surface between and/or around the balloons.

The various balloons may be configured to be independently and/or individually filled via multiple fill tubes 904. The fill tubes 904 may comprise one or more valves 914 configured to selectively control a direction of flow through the fill tubes 904. The device 903 may additionally or alternatively comprise an inflatable bladder 918 configured to be separately and/or individually inflated via a fill tube 904. The one or more balloons may be disposed internally relative to the inflatable bladder 918. As the balloons and/or inflatable bladder 918 inflate, the device 903 may contract and/or may apply pulling force to the annulus and/or muscle to pull the annulus and/or muscle inwardly (e.g., towards a center point of the device 903.

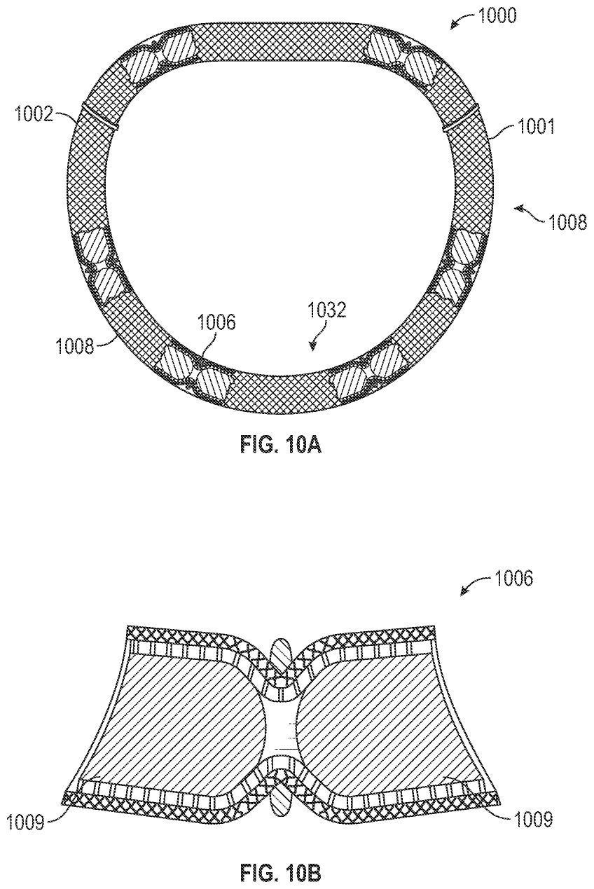

FIGS. 10A and 10B illustrate an example valve repair device 1000 in accordance with one or more examples. FIG. 10A provides a partially cross-sectional top view of the device 1000 with cross sections through a covering 1001 to allow viewing of one or more flexible joints 1006 of the device 1000. FIG. 10B provides a close-up view of a joint 1006.

In some examples, the device 1000 may comprise a distensible annuloplasty ring with expandable segments 1008 to accommodate an annulus of a valve. If the device 1000 is installed undersized, segments 1008 may be expanded (e.g., off-pump) in a beating heart. The increased flexibility of the device 1000 may allow for slight error in sizing pre-delivery.

The device 1000 may be at least partially expandable and/or the joints 1006 and/or segments 1008 may be at least partially flexible. In some example, the segments 1008 may be coupled end-to-end by flexible sheaths and/or joints 1006 enabling relative translation movement. In addition to providing stability and support, these elastic (semi-rigid) portions allow for a degree of annular expansion. In some examples, the segments 1008 may form a ring 1002. The ring segments 1008 coupled together may be relatively movable with respect one another so that the ring 1002 may expand to accommodate expansion.

The ring 1002 is made up of segments 1008 that are linked together with flexible joints 1006 forming the body in a shape to fit a valve (e.g., mitral valve) annulus. When cardiac pressure is applied and exerted against the ring 1002, the joints 1006 may stretch and/or expand the ring 1002 in a circumferential direction. The flexible joints 1006 may be made of one or more shape-memory materials that can return to a pre-determined shape. Example materials can include silicone, polyurethane, expanded polytetrafluoroethylene, etc.

While the ring 1002 is shown comprising six segments 1008, the ring 1002 can be composed of any number of segments 1008. The segments 1008 may be formed from any suitable and/or biologically compatible material. A length of each segment 1008 may be dependent on the shape and/or geometry of the ring 1002.

The joints 1006 can be designed with varying and/or pre-determined degrees of flexibility. In some examples, flexibility of a joint 1006 may be determined based at least part on a location of the joint 1006. For example, joints 1006 at or near a posterior annulus portion 1032 of the device 1000 may have greater flexibility than other joints 1006. Bonding between the segments 1008 and/or joints 1006 can be accomplished with, for example, solvent bonding, ultraviolet (UV) light curing, epoxy resins, and/or ultrasonic welding.

The ring 1002 may be covered at least partially with a covering 1001 comprising one or more stretchable fabrics that can accommodate the expansion of the ring 1002. In some examples, the covering 1001 can serve as a sewing cuff. The covering 1001 can be composed of any suitable materials, which can include Dacron and/or polyester.

The ring 1002 can have any suitable size and/or shape. In some examples, the ring 1002 may be C-shaped and/or may not form a complete ellipse. In other examples, the ring 1002 may have a closed and/or elliptical shape (e.g., D-shaped and/or saddle-shaped).

The one or more joints 1006 may be configured to adjust the circumference and/or shape of the ring 1002. In some examples, the segments 1008 can comprise a core 1009 configured to be bonded to the joints 1006 (e.g., fit at least partially into the joints 1006). The core 1009 can be composed of any suitable material(s), which can include silicone and/or metal. The core 1009 can have a flexible, semi-rigid, and/or generally rigid structure. The cores 1009 of the various segments 1008 may be disconnected from each other to allow angle and/or distance displacement relative to each other.

In some examples, the one or more joints 1006 may be at least partially expandable joints to increase a size of the ring 1002 and/or to accommodate a changing shape of the valve annulus throughout the cardiac cycle. The joints 1006 may be configured to allow for ring expansion. For example, the joins 1006 may be configured to allow the ring 1002 to be sized up to various predetermined sizes in a step-like manner. In some examples, the device 1000 may comprise one or more latching mechanisms to hold the device 1000 and/or ring 1002 in various predetermined sizes. In this way, the device 1000 may advantageously be configured to restore a valve to a more functional state by providing expansion and/or flexibility where it is needed that furthers improve leaflet coaptation to reduce valve regurgitation.

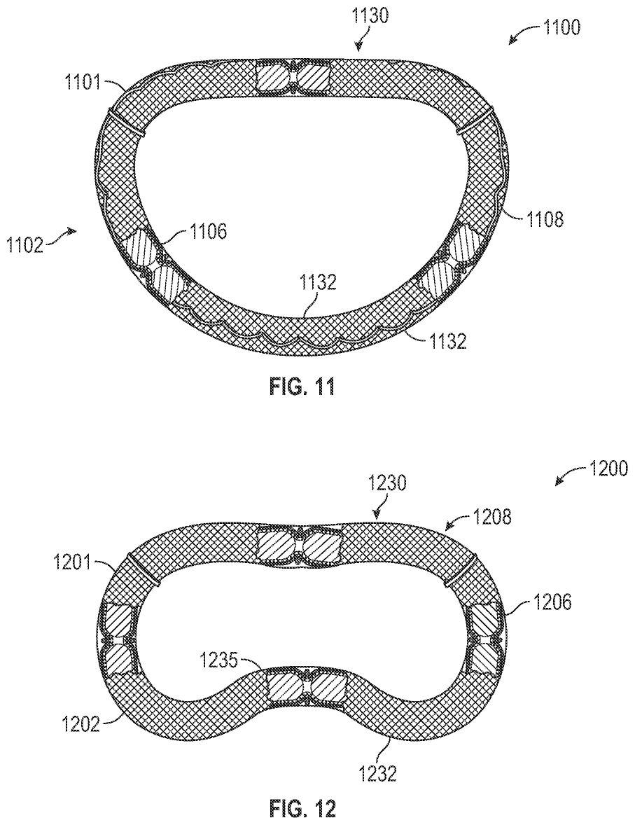

FIG. 11 illustrates at least a portion of an example valve repair device 1100 comprising a ring 1102 and/or ring-shaped expandable device in accordance with one or more examples. As shown in FIG. 11, the device 1100 can have a closed and/or D-shaped form, in which a posterior annulus portion 1132 of the device 1100 is generally curved and/or an anterior annulus portion 1130 is generally straight and/or less curved than the posterior annulus portion 1132. The device 1100 can comprise a covering 1101 at least partially enclosing one or more segments 1108 and/or joints 1106 of the device 1100. The device 1100 can comprise any number and/or position of joints 1106. In the example shown in FIG. 11, the device 1100 comprises one joint 1106 at the anterior annulus portion 1130 and an additional two joints 1106 along the posterior annulus portion 1132 of the ring 1102, with the joints 1106 disposed approximately equidistant from a center line of the posterior annulus portion 1132 bisecting the ring 1102.

FIG. 12 illustrates at least a portion of an example valve repair device 1200 comprising a ring 1202 and/or ring-shaped expandable device in accordance with one or more examples. As shown in FIG. 12, the device 1200 can have a closed and/or oval-shaped form, in which a posterior annulus portion 1232 of the device 1200 comprises a bend and/or an anterior annulus portion 1230 is generally straight. The device 1200 can comprise a covering 1201 at least partially enclosing one or more segments 1208 and/or joints 1206 of the device 1200. The device 1200 can comprise any number and/or position of joints 1206. In the example shown in FIG. 12, the device 1200 comprises two generally in-line joints 1206 along a minor axis of the device 1200, with a first joint 1206 at the posterior annulus portion 1232 and another joint 1206 at the anterior annulus portion 1230. The device 1200 further comprises two generally in-line joints 1206 along a major axis of the device 1200. In some examples, the posterior annulus portion 1232 may bend towards the anterior annulus portion 1230 and/or may bend orthogonally to the anterior annulus portion 1230 such that a bent portion 1235 of the posterior annulus portion 1232 is raised and/or lowered to a different plane than the anterior annulus portion 1230.

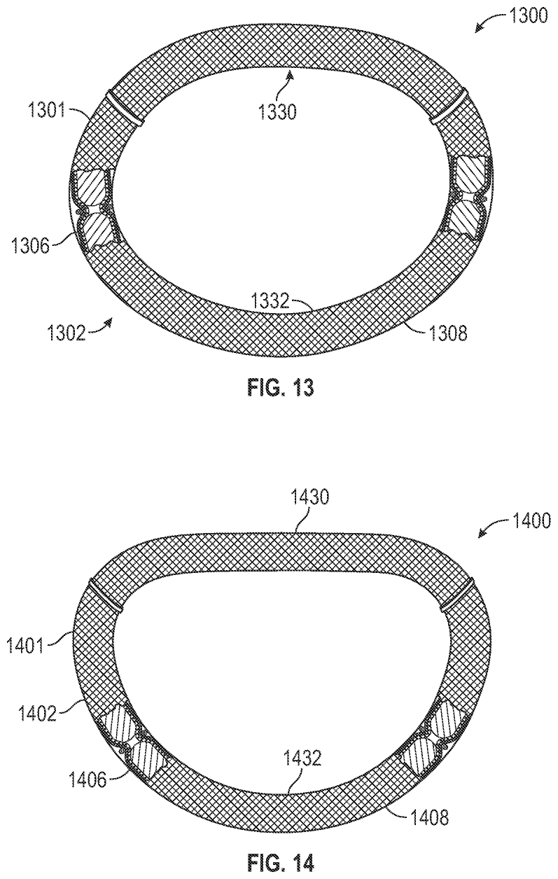

FIG. 13 illustrates at least a portion of an example valve repair device 1300 comprising a ring 1302 and/or ring-shaped expandable device in accordance with one or more examples. As shown in FIG. 13, the device 1300 can have a closed and/or oval-shaped form, in which a posterior annulus portion 1332 of the device 1300 is generally curved and/or an anterior annulus portion 1330 is generally curved and/or less approximately equally curved relative to the posterior annulus portion 1332. The device 1300 can comprise a covering 1301 at least partially enclosing one or more segments 1308 and/or joints 1306 of the device 1300. The device 1300 can comprise any number and/or position of joints 1306. In the example shown in FIG. 13, the device 1300 comprises one joint 1306 at a first side between the anterior annulus portion 1330 and the posterior annulus portion 1332 and another joint 1306 generally in-line and/or opposite the first joint 1306 at a second side between the anterior annulus portion 1330 and the posterior annulus portion 1332.

FIG. 14 illustrates at least a portion of an example valve repair device 1400 comprising a ring 1402 and/or ring-shaped expandable device in accordance with one or more examples. As shown in FIG. 14, the device 1400 can have a closed and/or D-shaped form, in which a posterior annulus portion 1432 of the device 1400 is generally curved and/or an anterior annulus portion 1430 is generally straight and/or less curved than the posterior annulus portion 1432. The device 1400 can comprise a covering 1401 at least partially enclosing one or more segments 1408 and/or joints 1406 of the device 1400. The device 1400 can comprise any number and/or position of joints 1406. In the example shown in FIG. 14, the device 1400 comprises two joints 1406 along the posterior annulus portion 1432 of the ring 1402, with the joints 1406 disposed approximately equidistant from a center line of the posterior annulus portion 1432 bisecting the ring 1402.

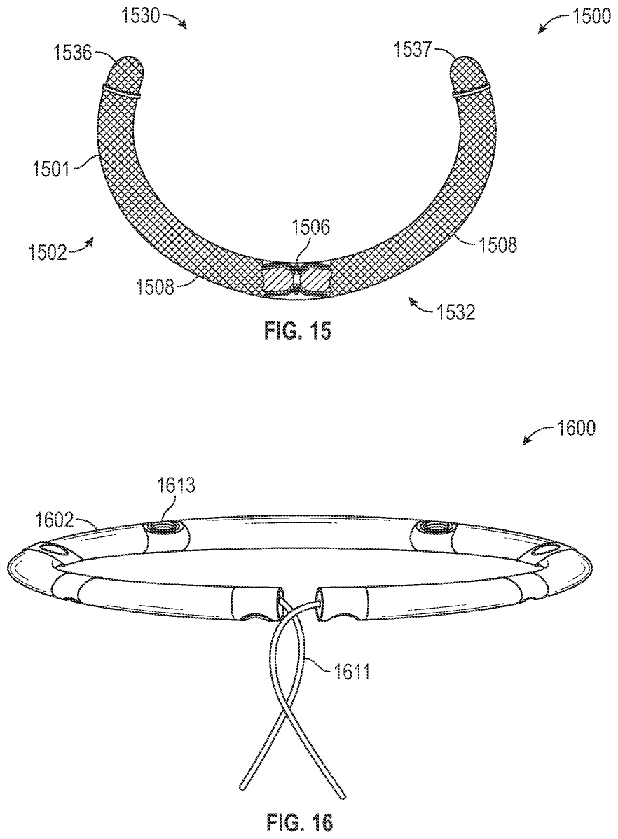

FIG. 15 illustrates at least a portion of an example valve repair device 1500 comprising a ring 1502 and/or ring-shaped expandable device in accordance with one or more examples. As shown in FIG. 15, the device 1500 can have an open and/or C-shaped and/or horseshoe form, in which a posterior annulus portion 1532 of the device 1500 is generally curved and/or an anterior annulus portion 1530 is disconnected and/or comprises a first end 1536 and/or a second end 1537. The device 1500 can comprise a covering 1501 at least partially enclosing one or more segments 1508 and/or joints 1506 of the device 1500. The device 1500 can comprise any number and/or position of joints 1506. In the example shown in FIG. 15, the device 1500 comprises a single joint 1506 at a midpoint of the posterior annulus portion 1532.

FIG. 16 illustrates another example valve repair device 1600 in accordance with one or more examples. The device may be configured to provide an ability to externally adjust the device 1600 (e.g., annuloplasty ring) in a beating heart to fine tune accurate sizing. The adjustment of the device 1600 can be made through tensioning one or more cords 1611 extending outside the heart and/or accessible to a surgeon. The cords 1611 can operate similarly to drawstrings and/or can be configured to cinch a ring 1602 of the device 1600 when tensioned and/or pulled.

The device 1600 may be configured to be adjustable off-pump and/or in a beating heart. The one or more cords 1611 may be accessible externally through, for example, a ventricular and/or atrial wall to allow for adjustment to the size and/or shape of the ring 1602 to cause reshaping of the annulus. The ring 1602 may be adjusted symmetrically by pulling both cords 1611 and/or cord ends (e.g., purse-string-suture ends) equally. Moreover, the ring 1602 may be adjusted asymmetrical by pulling one cord 1611 end more than the other.

The ring 1602 may comprise one or more openings 1613 configured to receive one or more tissue anchors. The rings 1602 can have any number of openings 1613. In some examples, the device 1600 can comprise two or more openings 1613.

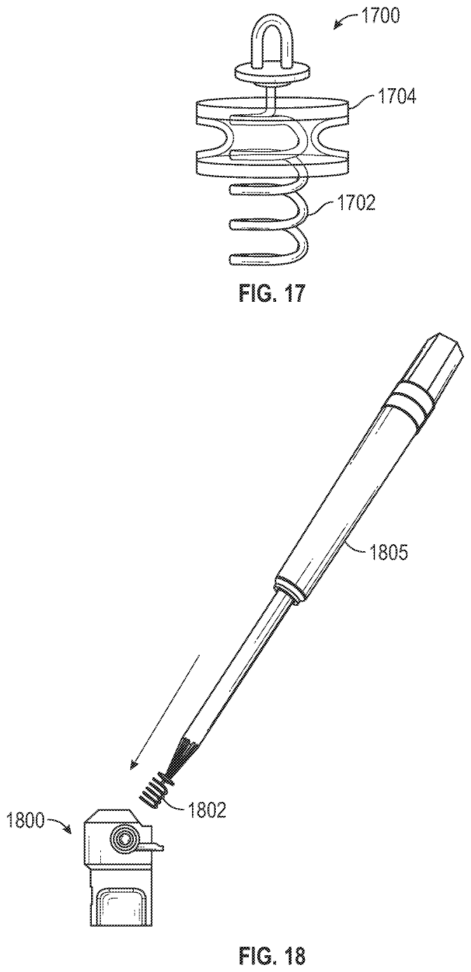

FIG. 17 illustrates an example tissue anchor 1700 configured to anchor one or more valve repair devices described herein. In some examples, an anchor 1700 may be configured to be driven into an opening of a ring of a repair device. The anchor 1700 may comprise one or more screws 1702 and/or a locking mechanism 1704.

FIG. 18 illustrates an example process of driving a tissue anchor 1802 into a repair device 1800 via an anchor driver 1805 in accordance with one or more examples. The driver 1805 may be configured to be held by a surgeon and/or may be configured for delivery and/or tensioning of one or more anchors 1802. The driver 1805 and/or anchor 1802 may be twisted in a first direction (e.g., clockwise) to cause reduction in size of a repair device and/or may be twisted in a second direction (e.g., counterclockwise) to cause an increase in size of the repair device.

The driver 1805 and/or anchors 1802 may be configured to enhance surgical procedures by providing the surgeon more control to make fine adjustments and achieve the desired annular dimension of a repair device (e.g., annuloplasty ring) and/or to restore a valve to a more functional state and/or facilitate leaflet coaptation to reduce valve regurgitation. In some examples, confirmation of regurgitation reduction can be confirmed off-pump in a beating heart and/or under echo guidance.

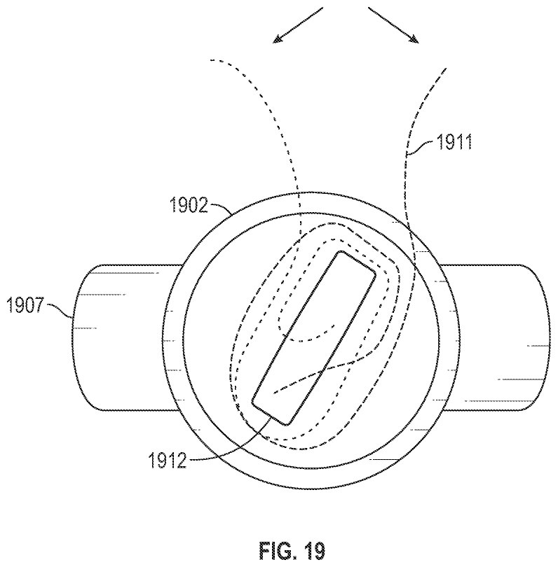

FIG. 19 illustrates one or more cords 1911 (e.g., implant contraction wires) extending into an example tissue anchor 1902 and/or clip in accordance with one or more examples. The one or more cords 1911 may be configured to facilitate cinching of a ring of a repair device as described in other examples herein and/or may be configured to form artificial chords extending from a prosthetic valve and/or valve repair device. The one or more cords 1911 may be composed of any suitable material(s), which can include a mesh of nickel titanium wire. In some examples, the anchor 1902 may be configured to be driven into contact with a base portion 1907 (e.g., pledget) configured to provide a firm backing for the anchor 1902. The base portion 1907 may be configured to be pressed into contact with native tissue (e.g., an atrial wall).

The anchor 1902 may comprise one or more openings 1912 (e.g., cavities) configured to receive the one or more cords 1911 and/or a delivery tool (e.g., anchor driver). A tensioning device may be inserted at least partially into the opening 1912 to tighten and/or pull the cords 1911.

A delivered repair device may be composed of any suitable materials, which can include, for example, a polyester sleeve, stainless steel anchors 1902, and/or one or more contraction cords 1911 and/or wires. The sleeve may be attached to tissue anchors 1902 connected to one or more cords 1911 encased in a delivery system.

Prior to a procedure, a patient may undergo a preprocedural examination and/or cardiac computed tomography (CT) to assess anatomical feasibility. Computed tomography scanning can be used to size the mitral annulus and/or to plan the procedure.

During the procedure, the surgeon can determine a starting point on the annulus. An implant (e.g., repair device) may be placed on the desired location. A first anchor 1902 may be implanted through the polyester sleeve and/or into the annulus tissue. The anchor 1902 may be released after proper anchoring is checked with, for example, push-and-pull testing. The surgeon can then move to the next anchoring point along the posterior annulus. These steps can be repeated to complete the implant around the annulus or a section of the annulus. Once the implant is completed, the implant is disconnected from the delivery system.

In some examples, ends of the cords 1911 may be pulled through the anatomy (e.g., atrium). The ends may be connected to a tensioner (e.g., anchor driver) that may be secured to the anatomy (e.g., atrium). The anatomy (e.g., atrium) may be closed and/or the patient may be taken off by-pass.

Under transesophageal echocardiographic (TEE) guidance, the one or more cords 1911 may be tensioned (e.g., with an adjustment tool) to cinch the annulus until the desired appropriate annular size has been reached.

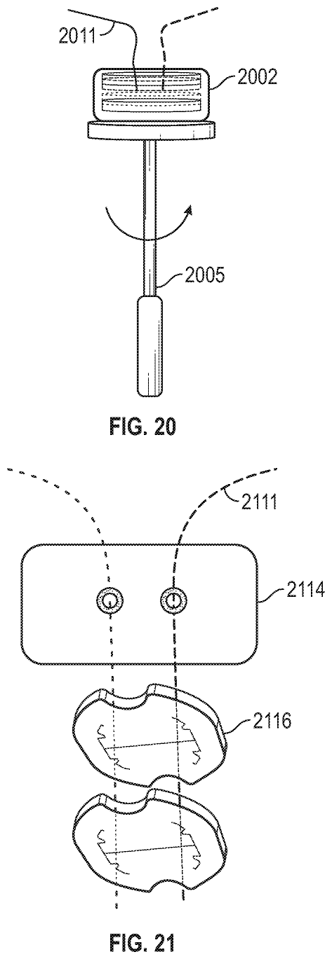

FIG. 20 illustrates driving of one or more tissue anchors 2002 using an anchor driver 2005 in accordance with one or more examples. The anchor driver 2005 may be configured to be twisted to cause tensioning and/or untensioning of one or more cords 2011 extending through the anchor 2002.

FIG. 21 illustrates anchoring of one or more cords 2111 using a pledget 2114 and/or one or more clips 2116. The pledget 2114 may be configured to provide a firm backing for the clips 2116. For example, the pledget 2114 may be placed against a surface of a tissue wall and/or the clips 2116 may be placed against the pledget 2114.

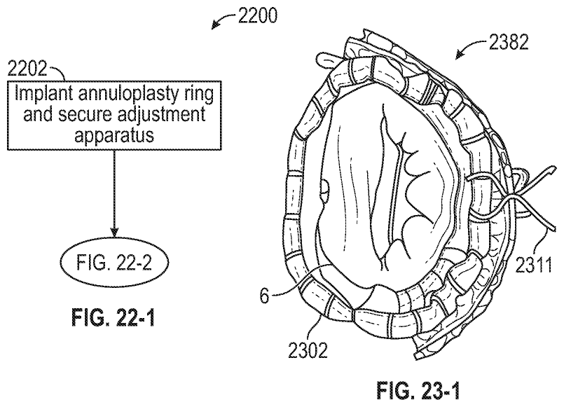

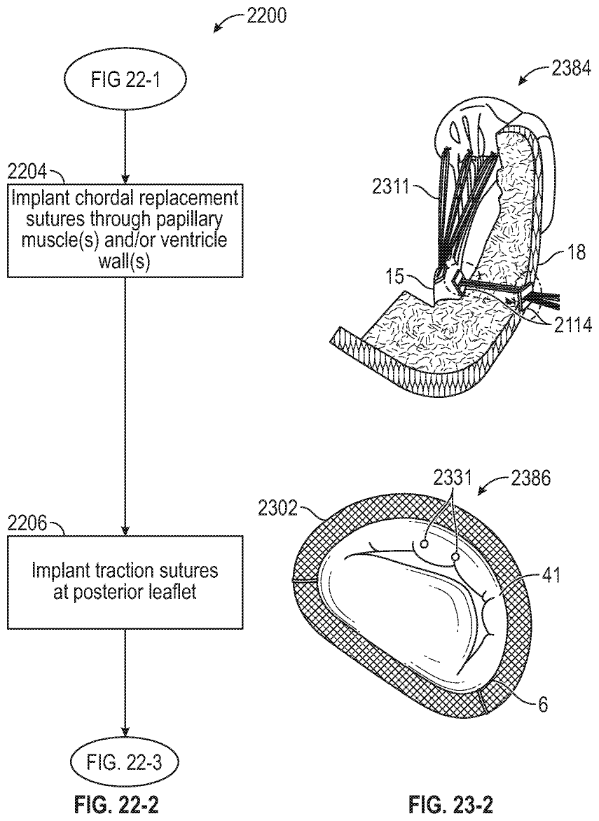

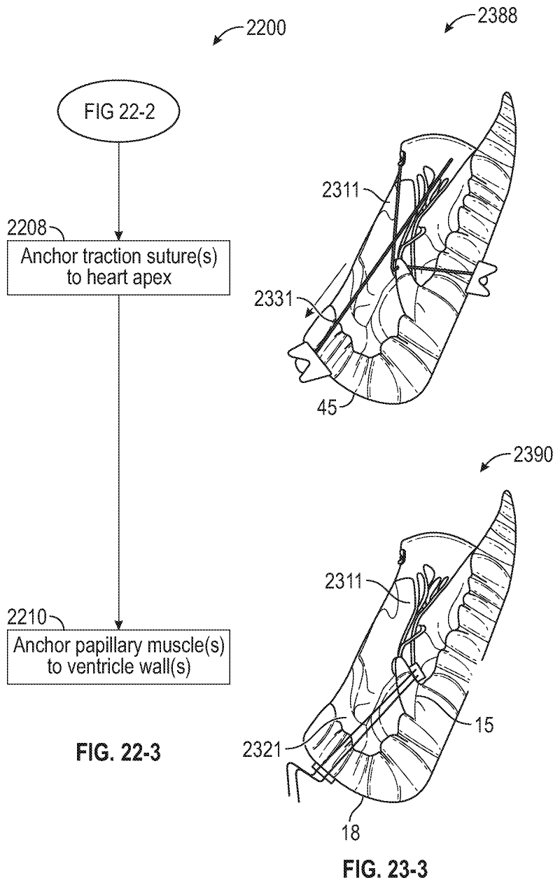

FIG. 22 (FIGS. 22-1, 22-2, and 22-3) is a flowchart illustrating steps of a process 2200 for delivering and/or deforming a valve repair device in accordance with one or more examples of the present disclosure. FIG. 23 (FIGS. 23-1, 23-2, and 23-3) provides certain images depicting various features associated with steps of the process 2200 depicted in FIG. 22. Adjustable chordal tensioning allows for titration and re-tension to improve leaflet coaptation after implant.

The process 2200 may comprise various preliminary steps, which can include one or more incisions (e.g., at the left atrium), identifying landmarks, and/or placing one or more sutures around an annulus of a target valve.

At step 2202, the process 2200 involves implanting an annuloplasty ring 2302 and/or other repair device at the valve 6 and/or securing one or more cords 2311, tubes, and/or other adjustment apparatus to the ring 2302 and/or anchors, as shown in image 2382 of FIG. 23. In some examples, the ring 2302 and/or implant may be secured on an atrial surface of an annulus of the valve 6. The various adjustment apparatus (e.g., drawstring and/or fill tube) may be secured to an external epicardial surface of an atrium.

In some examples, the ring 2302 may be parachuted to the annulus and sequentially tied via the various cords 2311 and/or traction sutures 2331. Various testing (e.g., hydrodynamic valve testing) may be used to evaluate repair of the valve 6.

At step 2204, the process 2200 involves implanting chordal replacement cords 2311 (e.g., artificial ePTFE sutures) beyond the valve 6 and/or through one or more papillary muscles 15 and/or ventricle walls 18 to replace ruptured or diseased native edge chords, as shown in image 2384 of FIG. 23. The cords 2311 may be configured to extend from the ring 2302 through head of the papillary muscle 15 and/or be secured at ends of the cords 2311 to an external surface of the ventricle (e.g., left ventricle). The one or more cords 2311 (e.g., cords) may be configured to extend through one or more pledgets 2114 in contact with the papillary muscle 15 and/or ventricle wall 18.

At step 2206, the process 2200 involves implanting one or more traction sutures 2331 at a posterior leaflet 41 of the valve 6, as shown in image 2386 of FIG. 23. The traction sutures 2331 may be implant at the posterior leaflet 41 close to the annulus of the valve 6. The traction sutures 2331 may be implanted midsegment to serve as accessory chords to further fine tune and improve repair of the valve 6.

The traction chords 2331 at the valve leaflets may be tensioned to pull and/or stretch a portion of the posterior leaflet 41 and/or a portion of the posterior annulus anteriorly and/or inwardly to effectively increase coaptation of the valve 6.

At step 2208, the process 2200 involves anchoring the traction sutures 2331 to an apex 45 portion of the heart, as shown in image 2388 of FIG. 23. In some example, the anchor points of the traction chords 2331 can be offset from the apex 45 of the heart to achieve a target force vector on the area of the leaflet 41. The applied tension at the traction suture(s) 2331 may be configured to draw the posterior leaflet 41 anteriorly.

One or more sutures may be anchored to the papillary muscle 15 and/or to the external ventricular surface apex 45 of the heart. Following anchoring of the sutures, the atrium may be closed and/or the patient may be taken off-pump. The annular ring may be adjusted using the sutures and/or fill tube on the epicardial surface, narrowing the size of the orifice or opening to achieve a desired physiologic effect. The length of the cords 2311 may be shorted and/or the cords 2311 may be tensioned to balance the cords 2311 to correct prolapse. In some examples, the traction sutures 2331 may be tensioned to pull the posterior leaflet 41 anteriorly to enhance the coaptation surface. In some examples, the cords 2311 may be pulled and/or tensioned to relocate the papillary muscle 15.

At step 2210, the process involves anchoring the papillary muscle 15 to one or more ventricle walls 18 (which can include the apex 45 region) using one or more sutures 2321, as shown in image 2390 of FIG. 23. The sutures 2321 may be configured to cause relocation of the papillary muscle 15 (e.g., posterior papillary muscle) to further enhance effective coaptation of the leaflet edges to further correct valve regurgitation.

In some examples, steps of the process 2200 may be performed in any order.

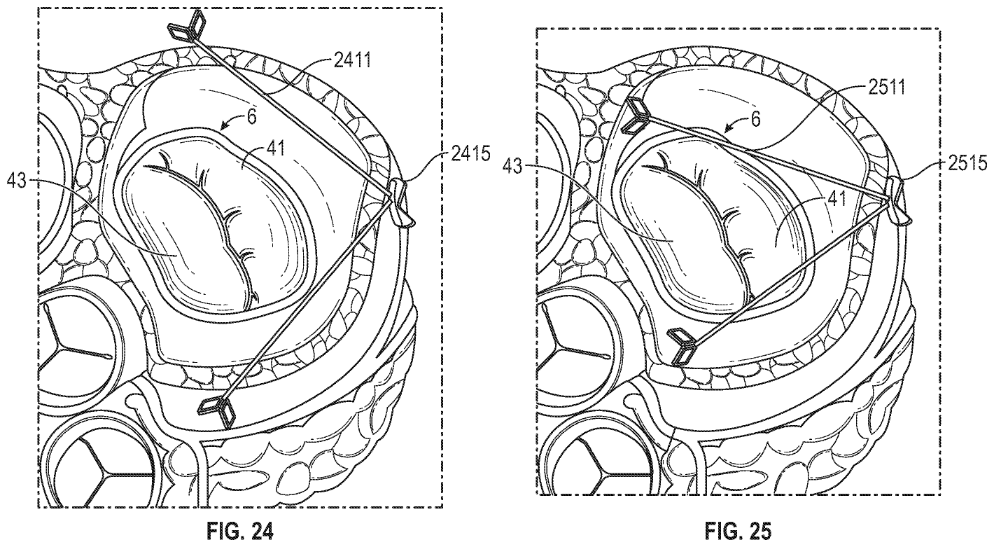

FIG. 24 illustrates an example method of suturing at or around an annulus of a valve 6 in accordance with one or more examples. The annulus of the valve 6 may be shaped in any of a variety of suturing techniques. For example, one or more implants 2415 (e.g., pledgets, clips, and/or anchors) may be sutured to the lateral and/or medial sides of the atrial wall via one or more cords 2411 to pull the posterior leaflet towards the anterior leaflet 43. The ends of the cords 2411 may be accessible externally for adjustment as the patient is taken off-pump. The suture anchors and/or implants 2415 can be located internally and/or possibly externally of the atrial wall. In FIG. 24, the implants 2415 are disposed externally of the atrial wall.

FIG. 25 illustrates an example method of suturing at or around an annulus of a valve 6 in accordance with one or more examples. The annulus of the valve 6 may be shaped in any of a variety of suturing techniques. For example, one or more implants 2515 (e.g., pledgets, clips, and/or anchors) may be sutured to the lateral and/or medial sides of the atrial wall via one or more cords 2511 to pull the posterior leaflet towards the anterior leaflet 43. The ends of the cords 2511 may be accessible externally for adjustment as the patient is taken off-pump. The suture anchors and/or implants 2515 can be located internally and/or possibly externally of the atrial wall. In FIG. 25, the implants 2515 are disposed internally to the atrial wall.

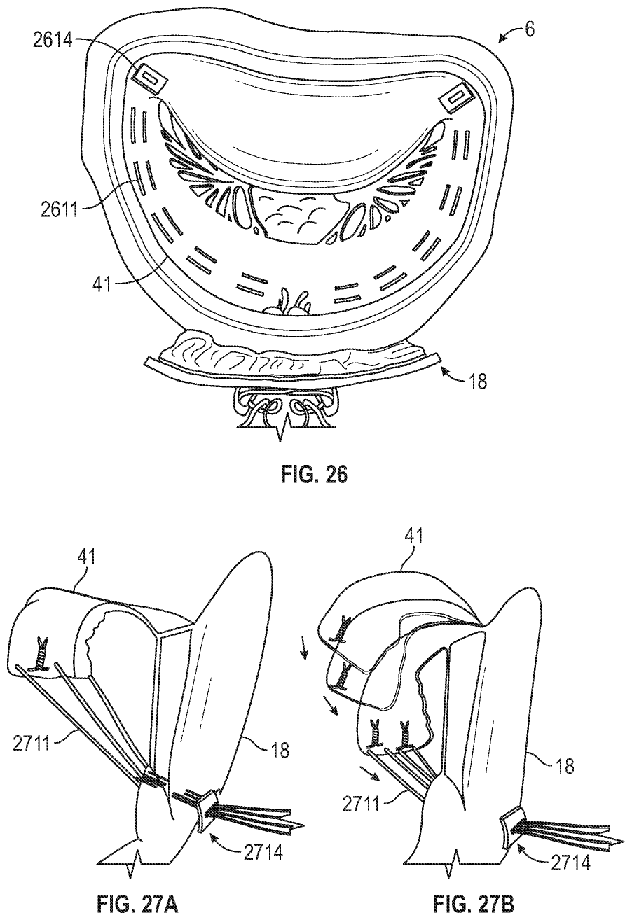

FIG. 26 illustrates a suturing technique at one or more leaflets 41 of a valve 6 in accordance with one or more examples. FIG. 26 provides a top view of the valve 6. One or more sutures 2611 may be stitched along a surface of the leaflets 41 (e.g., at or near an annulus portion of a heart valve). The sutures 2611 may be anchored to one or more anchors 2614 (e.g., pledgets, clips, and/or other devices) at the leaflet(s) 41. This suture annuloplasty technique can provide a simple, fast, and/or generally inexpensive repair process and/or can preserve flexibility of the annulus.

In some examples, the one or more sutures 2611 may be configured to extend from the valve 6 and/or through one or more ventricle walls 18. The one or more sutures 2611 may be configured to be anchored at an exterior surface of the ventricle wall 18 to provide anchoring for the sutures 2611. In some examples, the sutures 2611 may be sutured to a posterior leaflet 41 and/or may not be sutured to an anterior leaflet.

FIGS. 27A and 27B illustrate extension and/or titration of one or more cords 2711 extending from a leaflet 41 through a papillary muscle 15 and/or through a ventricle wall 18 in accordance with one or more examples. FIG. 27A illustrates initial placement of the cords 2711 and FIG. 27B illustrates an effect on the leaflet 41 of pulling the cords 2711, which may cause the leaflet 41 to pulled towards the papillary muscle 15 and/or ventricle wall 18. In some examples, the one or more cords 2711 may attach to and/or extend from an annuloplasty repair device as described herein and/or may be independently anchored to an annulus portion and/or leaflets 41 of a valve. In some examples, one or more pledgets 2714 and/or similar devices may be placed at exit and/or entry points of the cords 2711 and/or against an inner and/or outer surface of the ventricle wall 18.

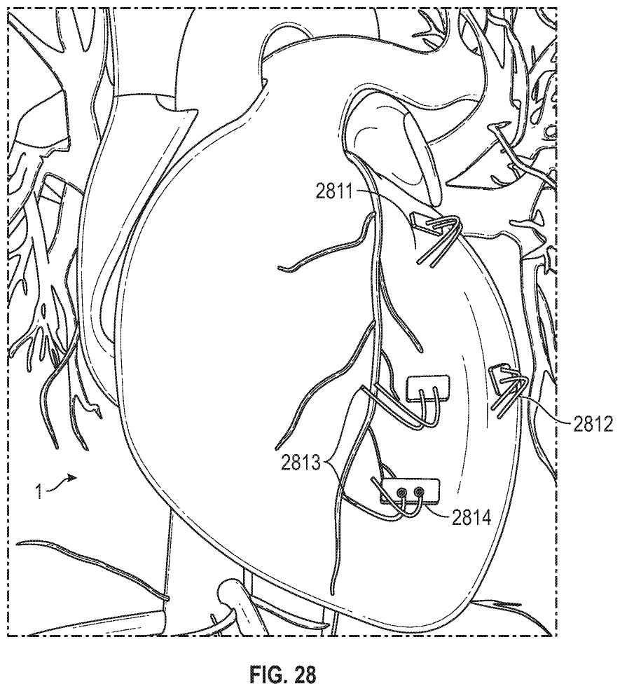

FIG. 28 provides an outer view of an example ventricle of a heart 1 with various sutures anchored at the walls of the ventricle in accordance with one or more examples. The various sutures can include papillary muscle relocation cords 2811, chordal adjustment sutures 2812, and/or traction adjustment sutures 2813. One or more sutures may attach to and/or extend from various repair devices described herein. In some examples, anchoring sutures may extend from a repair device and/or through one or more walls of the heart 1 to anchor to an outer surface of the heart 1. A repair system may additionally or alternatively comprise one or more anchors within the heart 1. For example, one or more sutures may be configure to pass through one or more papillary muscles and/or to secure to the one or more papillary muscles and/or an inner surface of the heart 1. As shown in FIG. 28, one or more pledgets 2814 and/or clips may be used at exit and/or entry points of the sutures to improve anchoring and/or to limit damage to the heart 1 from the sutures.

While the system of FIG. 28 is shown with four exterior anchor points at the external surface of the heart 1, example systems may utilize more or fewer anchoring points.

### Additional Features and Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.
Example 1: A device for treating a heart valve comprising: a curved frame; and a covering at least partially enclosing the curved frame.
Example 2: The device of any example herein, in particular example 1, further comprising a first expandable bladder connected to the curved frame.
Example 3: The device of any example herein, in particular example 2, further comprising a flow tube configured to allow inflow of a fluid into the first expandable bladder.
Example 4: The device of any example herein, in particular example 3, wherein the flow tube comprises a one-way valve.
Example 5: The device of any example herein, in particular example 2, wherein the curved frame is D-shaped and comprises a flat portion, and wherein the first expandable bladder is disposed across from the flat portion.
Example 6: The device of any example herein, in particular example 2, further comprising a second expandable bladder.
Example 7: The device of any example herein, in particular example 6, further comprising a first fill tube connected to the first expandable bladder and a second fill tube connected to the second expandable bladder.
Example 8: The device of any example herein, in particular example 1, further comprising one or more eyelets connected to an inner surface of the curved frame.
Example 9: The device of any example herein, in particular example 1, wherein the curved frame comprises two or more segments interconnected by one or more joints and configured to facilitate bending of the curved frame.
Example 10: The device of any example herein, in particular example 1, wherein the curved frame has a closed form.
Example 11: The device of any example herein, in particular example 1, wherein the curved frame has an open form.
Example 12: The device of any example herein, in particular example 1, wherein the curved frame comprises: one or more openings; and one or more tissue anchors within the one or more openings, the one or more tissue anchors comprising cavities.
Example 13: The device of any example herein, in particular example 1, further comprising a suture extending at least partially through the covering and comprising one or more free ends configured to extend outside the covering.
Example 14: A method comprising: delivering an implant to a valve of a heart, the implant comprising: a curved frame forming a closed loop; and a first suture; and anchoring the first suture to anatomy beyond the valve.
Example 15: The method of any example herein, in particular example 14, wherein the implant further comprises a first expandable bladder connected to the curved frame.
Example 16: The method of any example herein, in particular example 14, wherein the curved frame comprises: one or more openings; and one or more tissue anchors within the one or more openings, the one or more tissue anchors comprising cavities.
Example 17: The method of any example herein, in particular example 14, further comprising anchoring the first suture to a papillary muscle.
Example 18: The method of any example herein, in particular example 17, wherein the implant further comprises a second suture, and wherein the method further comprises anchoring the second suture to an apex region of the heart.
Example 19: The method of any example herein, in particular example 17, further comprising anchoring the first suture through the papillary muscle and to a ventricle wall of the heart.
Example 20: The method of any example herein, in particular example 17, further comprising anchoring the papillary muscle to a ventricle wall of the heart.

The above description of examples of the disclosure is not intended to be exhaustive or to limit the disclosure to the precise form disclosed above. While specific examples, and examples, are described above for illustrative purposes, various equivalent modifications are possible within the scope of the disclosure, as those skilled in the relevant art will recognize. For example, while processes or blocks are presented in a given order, alternative examples can perform routines having steps, or employ systems having blocks, in a different order, and some processes or blocks may be deleted, moved, added, subdivided, combined, and/or modified. Each of these processes or blocks can be implemented in a variety of different ways. Also, while processes or blocks are at times shown as being performed in series, these processes or blocks can instead be performed in parallel or can be performed at different times.

Certain terms of location are used herein with respect to the various disclosed examples. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

It should be understood that certain ordinal terms (e.g., “first” or “second”) can be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described can be performed in any suitable or desirable temporal order. Furthermore, any of the illustrated and/or described operations or steps can be omitted from any given method or process, and the illustrated/described methods and processes can include additional operations or steps not explicitly illustrated or described.

It should be appreciated that in the above description of examples, various features are sometimes grouped together in a single example, figure, or description thereof for the purpose of streamlining the disclosure and aiding in the understanding of one or more of the various aspects of the disclosure. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure should not be limited by the particular examples described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that can be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number can also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance can be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances can be more or less than a percentage level (e.g., dimension tolerance of less than approximately +1%). Some relativity between items can range from a difference of less than a percentage level to a few percent. Other relativity between items can range from a difference of a few percent to magnitude of differences.

One or more examples have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks can also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more examples are used herein to illustrate one or more aspects, one or more features, one or more concepts, and/or one or more examples. A physical example of an apparatus, an article of manufacture, a machine, and/or of a process can include one or more of the aspects, features, concepts, examples, etc. described with reference to one or more of the examples discussed herein. Further, from figure to figure, the examples can incorporate the same or similarly named functions, steps, modules, etc. that can use the same, related, or unrelated reference numbers. The relevant features, elements, functions, operations, modules, etc. can be the same or similar functions or can be unrelated.

What is claimed is:

1. A device for treating a heart valve comprising:
a curved frame; and
a covering at least partially enclosing the curved frame.

2. The device of claim 1, further comprising a first expandable bladder connected to the curved frame.

3. The device of claim 2, further comprising a flow tube configured to allow inflow of a fluid into the first expandable bladder.

4. The device of claim 3, wherein the flow tube comprises a one-way valve.

5. The device of claim 2, wherein the curved frame is D-shaped and comprises a flat portion, and wherein the first expandable bladder is disposed across from the flat portion.

6. The device of claim 2, further comprising a second expandable bladder.

7. The device of claim 6, further comprising a first fill tube connected to the first expandable bladder and a second fill tube connected to the second expandable bladder.

8. The device of claim 1, further comprising one or more eyelets connected to an inner surface of the curved frame.

9. The device of claim 1, wherein the curved frame comprises two or more segments interconnected by one or more joints and configured to facilitate bending of the curved frame.

10. The device of claim 1, wherein the curved frame has a closed form.

11. The device of claim 1, wherein the curved frame has an open form.

12. The device of claim 1, wherein the curved frame comprises:
one or more openings; and
one or more tissue anchors within the one or more openings, the one or more tissue anchors comprising cavities.

13. The device of claim 1, further comprising a suture extending at least partially through the covering and comprising one or more free ends configured to extend outside the covering.

14. A method comprising:
delivering an implant to a valve of a heart, the implant comprising:
a curved frame forming a closed loop; and
a first suture; and
anchoring the first suture to anatomy beyond the valve.

15. The method of claim 14, wherein the implant further comprises a first expandable bladder connected to the curved frame.

16. The method of claim 14, wherein the curved frame comprises:
one or more openings; and
one or more tissue anchors within the one or more openings, the one or more tissue anchors comprising cavities.

17. The method of claim 14, further comprising anchoring the first suture to a papillary muscle.

18. The method of claim 17, wherein the implant further comprises a second suture, and wherein the method further comprises anchoring the second suture to an apex region of the heart.

19. The method of claim 17, further comprising anchoring the first suture through the papillary muscle and to a ventricle wall of the heart.

20. The method of claim 17, further comprising anchoring the papillary muscle to a ventricle wall of the heart.
